(19) United States
(12) Patent Application Publication
(10) Pub. No.: US 2006/0253084 A1
(43) Pub. Date: Nov. 9, 2006

(54) Outdwelling slit valves and assemblies for medical liquid flow through a cannula and related methods

(76) Inventor: Greg Nordgren, North Logan, UT (US)

Correspondence Address: LYNN G. FOSTER, 602 East 300 South, Salt Lake City, UT, 84102, US

(21) Appl. No.: 11/121,342

(22) Filed: May 3, 2005

(51) Int. Cl. A61M 5/00

(52) U.S. Cl. 604/247

(57) ABSTRACT

Novel outdwelling nipple-shaped slit valves and outdwelling slit valve assemblies for influent and effluent fluid flow into and from a medical patient are disclosed, as well as related methods.

### FIELD OF INVENTION

The present invention relates generally to outdwelling control of medical liquid flow in a cannula and, more particularly, to novel normally closed outdwelling slit valves and slit valve assemblies and related methods for selective slit valving of medical liquid flow, including but not limited to bi-direction flow, along a hollow cannula, such as a catheter tube or needle.

### BACKGROUND

In the past, slit valves have traditionally been used in the side walls of otherwise closed indwelling catheter tubes to infuse or aspirate fluid. Use of such side wall slit valves has been directed to infusion and aspiration of liquids in the cardiovascular systems of medical patients, infusion and aspiration of fluids in the respiratory systems of medical patients, and infusion and aspiration in other body cavities.

Disadvantageously, sometimes the central passageway within an indwelling catheter tube comprising one or more side wall slit valves is partially or totally occluded when the slit valve is flexed inwardly from its normally closed position to an open position. Also, interference can occur between the lips of the slit, as they are flexed outwardly, and the wall of the body cavity in which the catheter tube and indwelling slit valve are disposed, which either prevents the slit valve from opening or undesirably limits the extent to which it is permitted to open or prevents or unduly limits flow.

Outdwelling slit valves have been proposed in the past. For example, see U.S. Pat. Nos. 5,201,722 and 5,984,902, which disclose transversely directed disc-shaped slit valve diaphragms each having a central slit, the axial flexural displacement of which is mandatorily constrained by abutment structure fore and/or aft of each transverse disc-shaped diaphragm. Prior outdwelling slit valves leave unanswered problems of interior dead space and provision of greater rates of flow without compromising the level of back pressure.

### BRIEF SUMMARY AND OBJECTS OF THE INVENTION

In brief summary, the present invention overcomes or substantially alleviates past problems in the cannula-related slit valve field. One or more novel outdwelling nipple-shaped slit valves and outdwelling slit valve assemblies are provided, as well as related methods. The problems of dead space and increased flow rates without compromising back pressure are addressed by the present invention.

With the foregoing in mind, it is a primary object to overcome or substantially alleviate past problems in the cannula-related slit valve field.

Another paramount object is the provision of one or more novel outdwelling nipple-shaped slit valves, outdwelling slit valve assemblies and related methods.

A further valuable object is the provision of novel outdwelling slit valves, slit valve assemblies and related methods, which address the problems of dead space and increased flow rates without compromising the adequacy of back pressure.

These and other objects and features of the present invention will be apparent from the detailed description taken with reference to accompanying drawings.

### BRIEF DESCRIPTION OF THE DRAWINGS

FIG. 1 is a diagrammatic representation of one outdwelling slit valve assembly in accordance with the present invention for infusion and aspirating into and from an internal cavity of a medical patient;

FIG. 2 is a perspective of a hub, adaptor or two-part housing in which a slit valve, embodying principles in accordance with the present invention, is contained;

FIG. 3 is a cross-section taken along the lines of 3-3 of FIG. 2;

FIG. 4 is an exploded fragmentary cross-section of the assembly of FIG. 3;

FIG. 5 is an exploded perspective of the slit valve assembly similar to the one illustrated in FIG. 2;

FIG. 6 is a fragmentary exploded perspective of another split valve assembly, embodying principles of the present invention;

FIG. 7 is a fragmentary exploded perspective of still another slit valve assembly, in accordance with principles of the present invention;

FIGS. 8A and 8B are fragmentary cross-sections illustrating the manner in which a nipple-shaped slit valve of the present invention is expanded from a normally closed to an outwardly open position, accommodating influent fluid flow or infusion into a medical patient;

FIG. 8C is a fragmentary cross-section of the slit valve of FIG. 8B in an inwardly open position, accommodating effluent fluid flow or aspiration from a body cavity of the medical patient;

FIG. 9 is an enlarged perspective of the nipple-shaped slit valve of FIG. 5;

FIG. 10 is a fragmentary perspective of the nipple-shaped slit valve of FIG. 9, shown largely in cross section taken along line 10-10 of FIG. 9;

FIG. 11 is an enlarged perspective of the nipple-shaped slit valve of FIG. 7;

FIGS. 12 and 13 are fragmentary perspective of the nipple-shaped valve of FIG. 11, shown largely in cross section taken along lines 12-12 and 13-13 of FIG. 11, respectively;

FIG. 14 is an enlarged perspective of the nipple-shaped outdwelling slit valve of FIG. 6;

FIG. 15 is a fragmentary perspective of the nipple-shaped slit valve of FIG. 14, shown largely in cross section taken along lines 15-15 of FIG. 14;

FIG. 16 is an enlarged perspective of one more nipple-shaped outdwelling slit valve, embodying principles of the present invention;

FIG. 17 is a fragmentary perspective of the nipple-shaped slit valve of FIG. 16, shown largely in cross section taken along lines 17-17 of FIG. 16;

FIG. 18 is an enlarged perspective of a further outdwelling nipple-shaped slit valve of the present invention;

FIG. 19 is a fragmentary perspective of the nipple-shaped slit valve of FIG. 18, shown largely in cross section taken along lines 19-19 of FIG. 18;

FIG. 20 is an enlarged perspective of still another outdwelling nipple-shaped slit valve, in accordance with principles of the present invention; and

FIG. 21 is a fragmentary perspective of the nipple-shaped slit valve of FIG. 20, as shown largely in cross section taken along lines 21-21 of FIG. 20.

### DETAILED DESCRIPTION OF THE ILLUSTRATED EMBODIMENTS

The present invention solves or reduces past problems in the catheter-related slit valve field, applicable to the human respiratory system, the human circulatory system, and other body cavities. One or more normally closed nipple-shaped slit valves, which may be in a variety of forms, are disposed remote from the distal end of each catheter tube of a medical patient whereby problems of the past are greatly reduced, if not eliminated. The term outdwelling is used to mean placement of the slit valve at the proximal end of a cannula, the distal end of which is disposed within a body cavity for fluid flow purpose. Thus, the term outdwelling embraces both placement of the slit valve outside the body of a patient or placed subcutaneously at a non-cavity site for safety purposes only, such as in conjunction with an implanted port to control, at the proximal end of a cannula, the flow of liquid from the port to a body discharge site remote from the port and the slit valve. The slit valves, in proximal regions of catheter tubes, comprise one or more normally closed slit valves disposed in a two-part housing. The slit valves may be either one-way or two-way valves. By two-way, it is meant that a given proximal outdwelling slit-valve both aspirates and infuses fluid from and into the associated catheter tube or cannula. By one-way, it is meant a proximal outdwelling slit valve which either aspirates or infuses, but does not do both.

Accordingly, an outdwelling nipple-shaped slit valve may comprise from one normally closed slit up to several normally closed slits. One or more slits may be located in a rounded portion or in a flat region (a flat) of the nipple-shaped outdwelling slit valve, as deemed appropriate by those having skill in the art. It is not necessary that all slits have the same length or thickness. Some may extend into or across the apex of the nipple-shaped slit valve. The slits, in any of the nipple-shaped slit valves, may have a uniform thickness or a thickness which varies, as deemed most appropriate for an intended purpose.

The preferred materials for forming the nipple-shaped slit valve comprise silicone rubber, polyurethane and other suitable natural and synthetic elastomeric materials. The material at each slit valve must have sufficient flexibility for the lips forming the normally closed slit to flex inwardly or outwardly or both when predetermined pressure differentials are imposed thereon, in order to accommodate fluid flow in the direction desired. Treating the lips with a softening composition is known in the art and may take place to provide the desired flexibility.

The slits in the nipple-shaped slit valve may be parallel, perpendicular, staggered, radially disposed or otherwise oriented, as deemed most appropriate by those having skill in the art. Opposing or offset slit valves located on different sides of a lumen may be used.

The present invention provides catheter assemblies which may be inserted into a medical patient over a guide wire. Pressures required for infusion and aspiration may be lower than in the past. The nipple-shaped slit valves may be formed in any suitable fashion, zero pressure molding being one mode.

This invention comprises a slit valve located in a nipple-shaped hub attached at the proximal end of a catheter, the distal end of which is indwelling. For example, the catheter may be used to control fluid flow in and out of the body. Infusaids such as saline, blood, hyper alimentation, or any medication prescribed for a patient may be administered intravenously through the catheter. A paramount purpose of slit valves according to the present invention is to provide safety to the patient while the catheter is in use. Pressures inside and outside of the body can cause blood to enter the catheter which may clot or air in an open system to enter the body. Since the early 1980's valves of different types have been employed on the distal end (indwelling) of catheters and more recently certain types of proximal valves have been devised. Slit valves of the present invention have more flexibility to withstand pressures and allow greater controllability of the opening and closing of the slit valves during times of use and non-use when protection is needed.

Several configurations of this invention are disclosed. In all of these configurations there is at least one normally closed slit located in an outdwelling nipple-shaped slit valve. The location, length of each slit and wall thickness allow for variation in valve functions. For instance, the varying of the wall thickness and or slit length has an effect, to some extent, on opening pressure of the valve. The use of more than one slit valve in the same annular location will affect flows but not necessarily opening or closing pressures. Also, one slit which transverses two or more valve surfaces can impact opening pressures in one direction and not the other. Material properties also have an effect on the opening and closing of valves, i.e., if the material is soft, the valves will act different than if the material is of higher durometer. This creates the ability to adjust the configuration of the slit valve or valves and select a material to produce an effective valve for the purpose intended.

The prior art, such as U.S. Pat. Nos. 5,205,834 and 5,984,902, comprises a disc of material which has a slit of a certain length placed in the center of the disc. This disc is placed in annular compression within a hub. Valve function is varied by the opening diameter on either side of the disc. The disc can also be varied in thickness and material durometer which allows some flexibility in valve function. However, the length of the valve can only be increased by increasing the diameter of the disc itself. This “slit diameter” of disc with the area needed to hold the disc in place can create a large and bulky hub and makes it very difficult to make it small and compact. Also over pressurization of the system can dislodge the disc causing valve malfunction. In contrast, the present invention provides a very small and compact outdwelling slit valve. More valve slits along different surfaces of the valve allow reduction in valve size. It also allows the valve pressures to vary without restricting the diameters within the hub housing itself

The present invention accommodates the following:

1. Valves in flats only:
One or more slits can be placed on flat surfaces only to create valves. External surface and internal surface of flats may be different in that the internal flat will be smaller in area. Therefore, if the valve slit is in length of the external flat, the valve will by its nature have different operating pressures depending on whether it is opening outwardly or inward. If the valve slit is the length of the internal flat surface, the valve function will be identical inward or outward.

2. Valves on flat and wall:
One or more slits can be placed so as to transverse flat surfaces and the outer diameter and/or end surfaces to create the valves. Slits that extend beyond the flat valve surfaces will create a larger flow outwardly than inwardly. This creates a larger flow of fluid outward, therefore increasing flow rate or ease of infusion of fluid into the body.

3. Crossing valves:
One or more slits can be placed in a crossing orientation with one another or intersect one another. This also can create in effect a longer slit in the outwardly direction while maintaining a controlled inwardly slit length. Also, some crossing slits may not function at all in one direction, but when combined with another slit creates a larger opening for infusion.

4. Number of valves:
One or more slits can be on more than one surface creating many valves. The number of slit valves in each valve unit is the most significant feature, because each slit valve acts independently from the others. The more valve slits the higher combined infusion which is a plus. However, more slits do not effect the at rest valve functions which keeps internal and external pressures from opening the valves.

5. Wall thickness of valves:
Wall thickness can vary to change valve function. Wall thickness can be different on each valve as determined by those skilled in the art.

6. Length of slits:
Length of slits can vary to change valve function. The length of the slit has a large effect on valve function. Varying slit lengths could also be used as a way of controlling valve functions.

7. Flushing of dead space:
Dead space that cannot be flushed clean presents a problem in any aspiration and infusion system. The present slit valves expand to constrain the amount of dead space so that flushing keeps the interior clean and free of blood and other contaminates.

Reference is now made to the drawings, wherein like numerals are used to designate like parts throughout. Any normally closed nipple-shaped outdwelling slit valve of this invention may be used for infusing, aspirating or both and may be any one of many configurations. Each of the nipple-shaped slit valves shown in FIGS. 1-21 comprises one-piece construction, with each slit valve placed within an outdwelling housing positioned external of a medical patient in proximal relation to a hollow cannula, which selectively accommodates fluid flow to and from a medical patient.

FIG. 1 is intended to be representative of placement of outdwelling slit valves in accordance with the present invention external of the medical patient where a hollow cannula 30 comprising an open end 32 is disposed within a body cavity 34 of a medical patient 36. The slit valve of FIG. 1 is concealed within a housing, generally designated 38, comprising interconnected distal and proximal housing parts 40 and 42. The slit valve within housing 38 accommodates selective fluid flow into and from the patient 36, based upon the slit valve being subjected to a predetermined pressure differential, i.e., the pressure on the distal side in tube 30 when compared to the pressure on the proximal side in tube 44. While in most instances the body cavity 34 would likely be a cardiovascular vein, where the fluid flow comprises liquid displacement, liquid displacement in other body cavities is contemplated, as is gaseous flow from and to other body cavities.

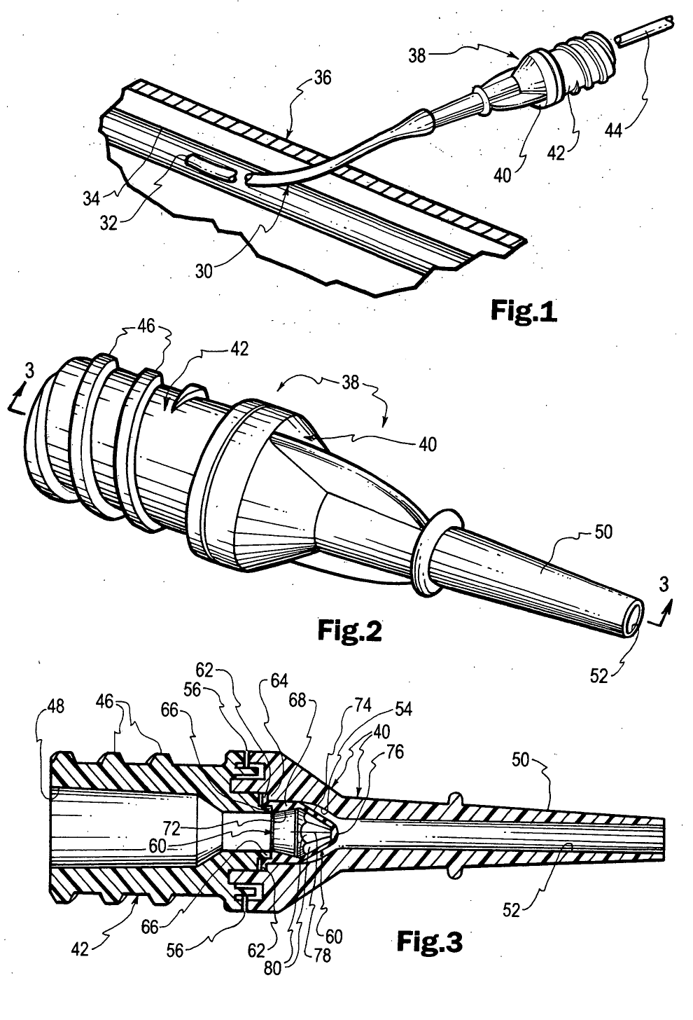

FIGS. 2-4 illustrate enlarged views of the two-part housing 38 of FIG. 1 and slit valve 60 within the housing 38. The exterior of the proximal housing part 42 comprises an exposed luer lock thread 46 and a hollow interior 48 to accommodate selective fluid flow. Thread 46 accommodates luer lock threaded connection with a luer lock fitting at the distal end of hollow proximal tube 44 (FIG. 1) in a conventional manner. Distal housing part 40 comprises a tapered elongated tip 50 sized to accommodate a press fit overlapping connected relationship with the proximal end of the cannula 30, the cannula 30 being illustrated as a hollow tube of synthetic resinous material of medical grade. The interior of the distal housing part 40 comprises the hollow passageway 52 accommodating selective fluid flow and an enlarged slit valve-receiving compartment 54.

The proximal and distal housing parts 42 and 40 are connected at interface 56 in an interlocking male-female relationship, with or without a bonding agent, as determined by those skilled in the art. Thus, when assembled as shown in FIG. 3, the housing parts 40 and 42 may be either separable or inseparable.

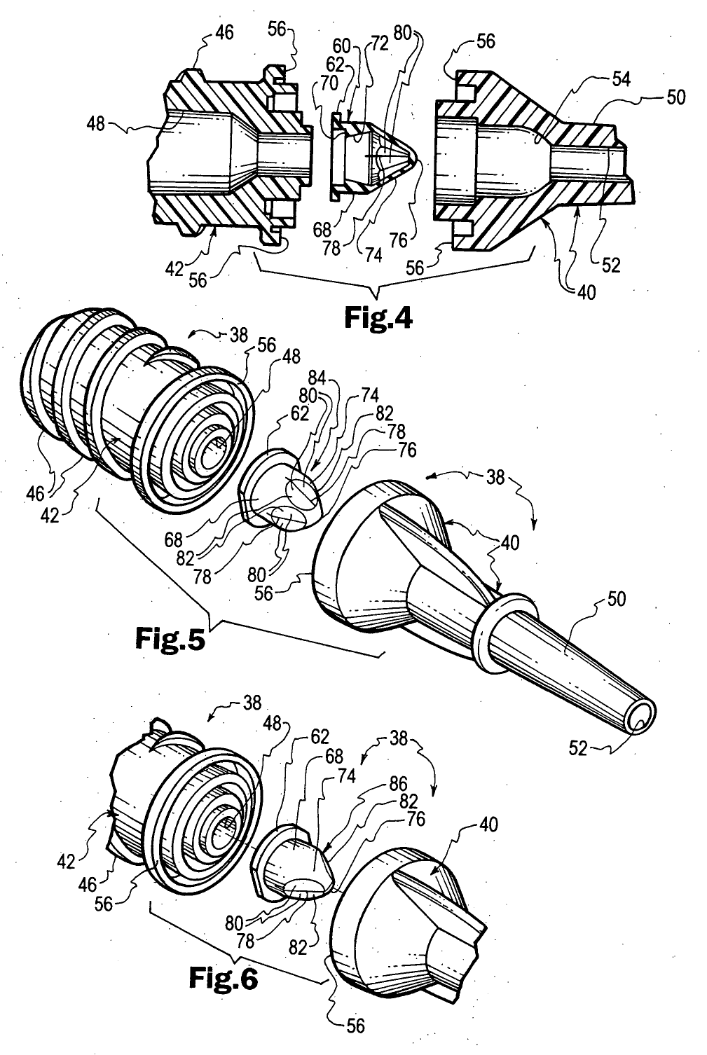

Contained in compartment 54, as shown in FIGS. 3 and 4, is a one-piece normally closed nipple-shaped slit valve, generally designated 60. The slit valve 60 comprises a proximal flange 62, which, when assembled, is compressively trapped between and compressively secured between the two housing parts 40 and 42, as best shown in FIG. 3. FIG. 3 illustrates that flange 62 is firmly retained between a distal shoulder 64 of housing part 40 and proximal shoulder 66 of housing part 42. While the central portion 68 of the slit valve 60 is annular at the outside, it comprises an interior shoulder 70, with the hollow 48 of valve 60, followed by a tapered interior surface 72, which is divergent in a distal direction.

The nipple-shaped slit valve 60 comprises a tapered distal section 74 ending in a tip or apex 76. The converging section 74 is equipped with at least one normally closed slit 78 comprised of opposed flexible lips 80. While the slit 78 may be placed in a flat region (a flat) of the slit valve, it may also be positioned in a rounded (or non-flat) portion of the slit valve. Where only one slit is used in the slit valve, that slit accommodates both influent and effluent fluid flow when predetermined pressure differentials are reached. In other embodiments, as explained hereinafter, nipple-shaped slit valves in accordance with the present invention may comprise separate influent and effluent slits. Influent flow is sometimes referred to as infusion and effluent is sometimes referred to as aspiration.

The nipple-shaped slit valve 84 of FIG. 5 is comprised of previously-described proximal flange 62, central portion 68 and a tapered portion 74, but comprised of four flats 82, each flat being equipped with a slit valve 78, each of which is spaced from the tip 76 but shown to extend proximally a short distance into the central portion 68. Any of the four slits 78 of nipple-shaped slit valve 84 are sized, shaped and formulated to accommodate infusion, while the other two slits accommodate aspiration, when in each case a threshold pressure differential is required to accommodate fluid flow, or any of the four slits could accommodate a two-way functioning valve.

With respect to FIG. 6, a somewhat different nipple-shaped outdwelling slit valve, generally designated 86, is illustrated which comprises the previously mentioned flange 62 and central portion 68 a distally extending convergingly shaped tapered distal portion and a tip 76. The nipple-shaped slit valve 86 comprises a single two-way slit which continuously traverses through two flats 82, across tip 76 and proximally beyond both flats 82. This slit accommodates both infusion and aspiration.

Reference is made to FIG. 7 which illustrates a nipple-shaped slit valve, in the form of a duckbill configuration, generally designated 88. Nipple-shaped slit valve 88 comprises the previously described flange 62 and intermediate or central hollow portion 68 as well as a distal portion 74′ defining opposed U-shaped flats 90, each comprising a slit 78 which extends proximally beyond the associated flat 90 into the central portion 68. The distal portion 78′ comprises a duckbill end 92 in which is defined a slit 78 running at both ends thereof into the distal cylindrical central portion of the slit valve 88. The two opposed slits 78 in flats 90 accommodate aspiration, while all three slits 78 accommodate infusion.

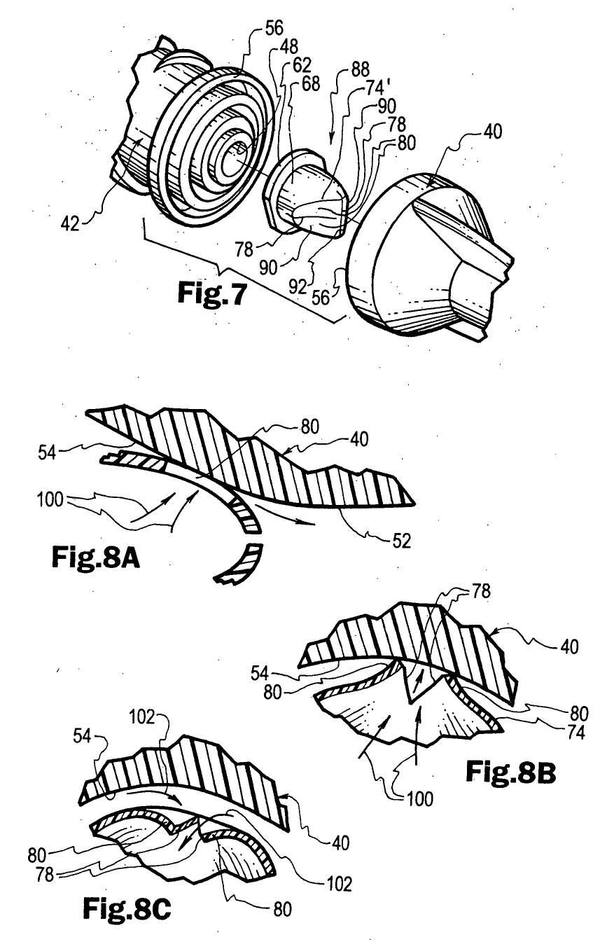

FIGS. 8A through 8C illustrate the manner in which the normally closed slits 78 are flexed at respective pressure differential thresholds to accommodate infusion and aspiration, respectively. Specifically, as shown in FIGS. 8A and 8B, infusion fluid, shown by arrows 100 reaches a pressure which compared to the pressure in passageway 52 and compartment 54 creates the necessary pressure differential threshold to accommodate opening of the slit 78 by flexing the lips 80 away from the normally closed contiguous relation to the open condition illustrated in FIG. 8B accommodating infusion flow at a selected rate. The rate may be controlled to less than a maximum by spacing the slit 78 in question in close proximity to the adjacent housing surface defining the compartment 54, as illustrated in FIG. 8A.

To the contrary, when the pressure within the passageway 52 and the compartment 54 exceeds the pressure within the interior of the nipple-shaped slit valve by an amount equal to or greater than the threshold differential pressure, the lips of the slit will flex inwardly as shown in FIG. 8C to accommodate aspiration in a proximal direction, as indicated by arrow 102.

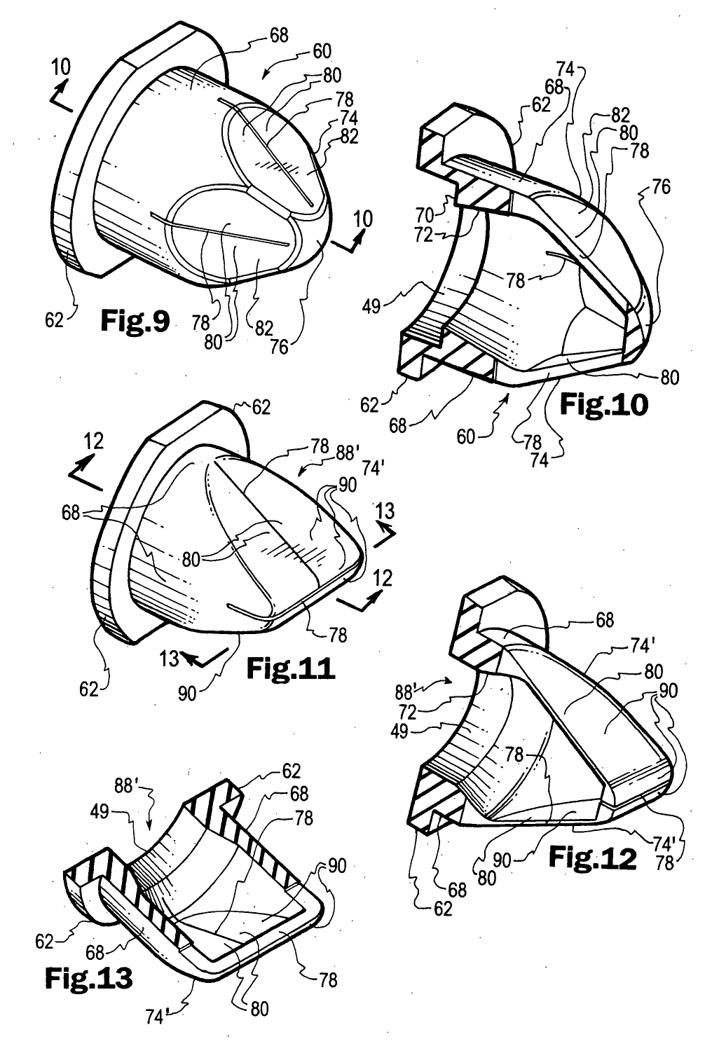

FIGS. 9 and 10 illustrate the above-described nipple-shaped slit valve 84 of FIG. 5, in enlarged presentation.

Reference is made to FIGS. 11 through 13, which illustrate a slight modification of the nipple-shaped slit valve 88 of FIG. 7. The embodiment of FIGS. 11 and 12 is generally 88′. The presentation in FIGS. 11 and 12 shows the slit valve 88 of FIG. 7 rotated by 90 degrees with the two slits 78 exposed in the U-shaped flats 90 terminating proximally within the flats so as not to extend into the cylindrical portion 68. It should be noted that the step or shoulder 70, described in conjunction with slit valve 60, has been replaced by solid material at the same location in the slit valve 88 of FIGS. 11-13.

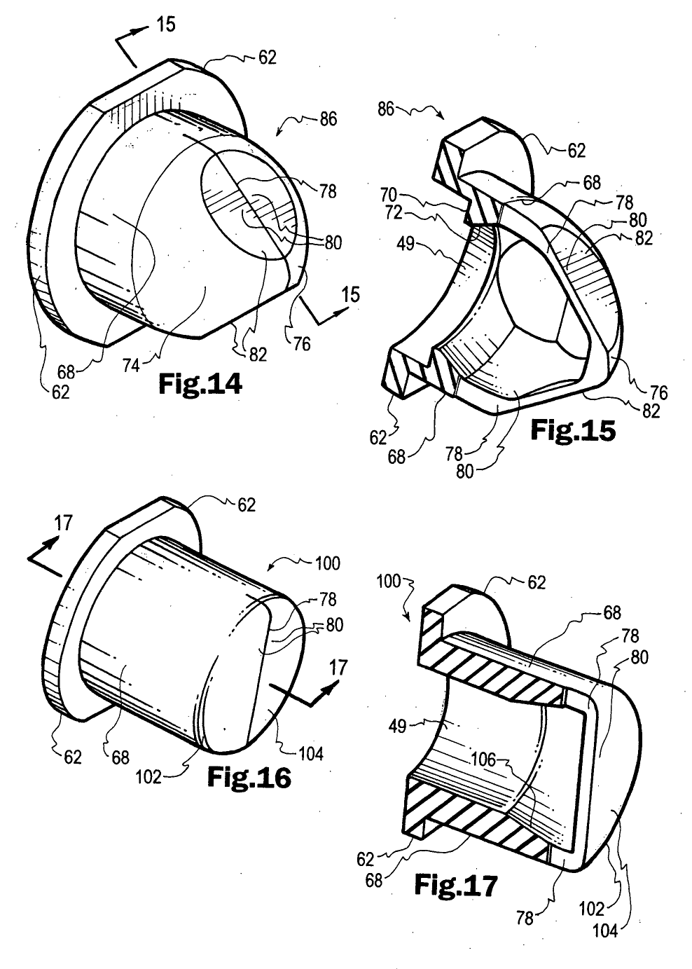

Reference is now made to FIGS. 14 and 15, which in enlarged form illustrate the previously described nipple-shaped slit valve 86 of FIG. 6. However, FIGS. 14 and 15 illustrate nipple-shaped slit valve 86 rotated through 90 degrees from the position of FIG. 6.

Reference is now made to FIGS. 16 and 17, which illustrate a further nipple-shaped slit valve, generally designated 100, in accordance with the principles of the present invention. The configuration comprises an extension of the cylindrical central region 68 and further comprises a blunt distal end 102 comprising a flat transversely-directed distal wall 104 and in which a single slit 78 exists so as to span the entire diameter of the distal end wall 104 and to further extend a short distance into the cylindrical portion 68, both top and bottom. In effect, the wall 104 comprises a flat, with the single valve 78 accommodating both infusion and aspiration. As can been seen from FIG. 17, the wall thickness of central cylindrical portion 68 decreases internally at tapered wall 106 so that the wall thickness is less at the distal region of the central portion 68 and across the end wall 104.

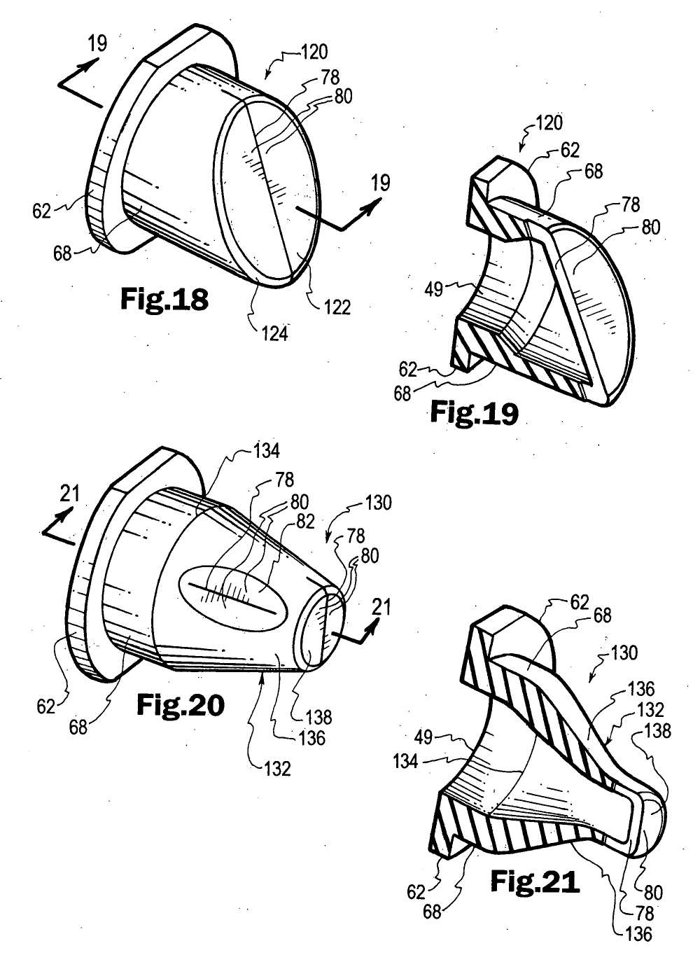

Reference is now made to FIGS. 18 and 19 which illustrate a further nipple-shaped outdwelling slit valve, generally designated 120, in accordance with principles of the present invention. Slit valve 120 comprises the previously described proximal flange 62 and hollow intermediate central cylindrical section 68. The distal end of the slit valve 120 comprises a diagonally-directed or beveled end wall 122 integrally joined at interface 124 to the cylindrical central section 68. End wall 122 comprises a normally closed slit 78, which extends centrally across the entire end wall 122 and a short distance, top and bottom, proximally into the cylindrical wall 68. Thus, the end-to-end length of the slit 78 of FIGS. 18 and 19 exceeds the diameter of the cylindrical wall 68, thereby accommodating substantial fluid flow when a pressure differential of a specific threshold or greater magnitude causes the lips 80 to flex away from each other and the normally closed slit 78 to open.

Reference is now made to FIGS. 20 and 21 which illustrate an additional nipple-shaped outdwelling slit valve, generally designated 130. Slit valve 130 comprises previously described proximal flange 62 and central hollow cylindrical section 68. A frusto-conical distal end segment 132 is integral, preferably as one piece, at interface 134 with cylindrical section 68.

The distal end segment 132 comprises a hollow frusto-conical wall 136 which tapers in a distal direction and in which at least one side flat 82 is disposed with a slit 78 thereon. The thickness of conical wall 136 decreases progressively in a distal direction. The wall 136 is closed by an integral blunt transversely directed end wall 138. Wall 138 defines a normally closed slit 78, which extends centrally across the entire diameter of end wall 138 and proximally into the wall 136, top and bottom.

The invention may be embodied in other specific forms without departing from the spirit of the central characteristics thereof. The present embodiments therefore are to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed and desired to be secured by Letters Patent is:

1. An outdwelling nipple-shaped one-piece proximal slit valve for use with a medical patient comprising:
a hollow proximal transverse slit valve flange;
a hollow nipple-shaped portion comprising:
an intermediate section connected to and distal of the flange and comprising a wall defining a hollow interior;
a tip distal of the intermediate section;
at least one normally closed slit located at least in part in the wall of the intermediate portion which slit selectively opens to accommodate fluid flow through the slit responsive to a predetermined pressure differential.

2. A slit valve according to claim 1 further comprising a retention shoulder disposed between the flange and the intermediate portion.

3. A slit valve according to claim 1 wherein the tip is slit-free.

4. A slit valve according to claim 1 wherein the tip contains a portion of at least one slit.

5. A slit valve according to claim 1 wherein the intermediate portion comprises at least one flat in which the at least part of one slit is located.

6. A slit valve according to claim 1 wherein the tip comprises at least one flat in which at least part of one slit is located.

7. A slit valve according to claim 1 wherein the at least one slit is selected from the group consisting of two, three and four slits.

8. A slit valve according to claim 7 wherein at least some of the two, three and four slits, respectively, are disposed at least in part in flats disposed in the intermediate portion wall.

9. A slit valve according to claim 7 wherein each of the slits comprise opposed normally closed flexible lips which selectively separate under the differential pressure to accommodate influent and effluent fluid flow.

10. A slit valve according to claim 7 wherein some of the slits comprise normally closed flexible lips which selectively separate to accommodate influent flow only and other slits comprise normally closed flexible lips which selectively separate to accommodate effluent flow only.

11. A slit valve according to claim 7 wherein at least a portion of at least one slit is disposed in the tip.

12. A slit valve according to claim 11 wherein the at least one tip slit comprises an extension of a slit partially disposed in the wall.

13. A slit valve according to claim 11 wherein the at least one tip slit is unconnected to any other slit.

14. A slit valve according to claim 1 wherein the tip is selected from the group consisting of a pointed configuration, a duck bill configuration and a blunt configuration.

15. A slit valve according to claim 1 wherein the wall of the intermediate portion is selected from the group consisting of a distally converging configuration and a non-converging configuration.

16. An outdwelling tapered one-piece nipple shaped proximal slit valve for use with a medical patient comprising:
a hollow proximal transverse slit valve flange;
a distal tip disposed distal of the flange;
a converging diagonally-disposed intermediate portion, interposed between the flange and the tip, comprising a distally converging diagonally-directed wall defining a hollow interior;
at least one diagonally-disposed normally closed slit located at least in part in the diagonally-directed wall which comprises normally closed lips which selectively separate responsive to a pre-set pressure difference to permit fluid flow.

17. An outdwelling proximal slit valve assembly for use with a medical patient comprising:
an outdwelling hollow housing comprising a proximal housing component interconnecting with a distal housing component;
an outdwelling nipple-shaped slit valve comprising a proximal slit valve end held within the hollow of the housing at the interconnect between the proximal and distal housing components;
the nipple-shaped slit valve comprising a hollow male element comprising a wall defining a hollow interior, the male element extending in a distal direction into the hollow of the distal housing component and ending in a distal tip also dispose in the hollow of the distal housing component;
at least one normally closed slit located at least in part in the wall which slit comprise lips which selectively open to accommodate fluid flow through the slit responsive to a predetermined pressure differential.

18. An assembly according to claim 17 wherein the outdwelling nipple-shaped slit valve is of one piece construction.

19. As assembly according to claim 17 wherein the slit valve lips when flexed to open the slip engage the adjacent distal housing component to limit the rate of fluid flow therethrough.

20. A method of providing to a medical patient an outdwelling slit valve for controlled fluid infusion and fluid aspiration, comprising the acts of:
providing a nipple-shaped slit valve comprising at least one normally closed slit which opens at a specific pressure differential to accommodate fluid flow;
placing the nipple-shaped slit valve in a hollow housing;
placing the slit valve-containing housing in flow accommodating relation with a hollow cannula at a site proximal of the medical patient;
placing a distal end of the cannula in a body cavity of the medical patient to accommodate fluid displacement, while retaining the slit valve and housing proximal of the patient.
